(19) United States
(12) Patent Application Publication
(10) Pub. No.: US 2015/0374875 A1
(43) Pub. Date: Dec. 31, 2015

(54) ABSORBENT ARTICLE COMPRISING A COOLING SYSTEM

(71) Applicant: The Procter & Gamble Company, Cincinnati, OH (US)

(72) Inventor: Raphael WARREN, Amberley Village, OH (US)

(21) Appl. No.: 14/731,961

(22) Filed: Jun. 5, 2015

Related U.S. Application Data
(60) Provisional application No. 62/018,734, filed on Jun. 30, 2014.

(51) Int. Cl. A61L 15/20; A61L 15/50; A61K 33/30; A61L 15/18; A61K 31/225; A61K 31/22

(52) U.S. Cl. CPC A61L 15/20; A61K 31/225; A61K 31/22; A61K 33/30; A61L 15/18; A61L 15/50; A61F 13/51113

(57) ABSTRACT

An absorbent article comprising a cooling system, where the cooling system comprises one or more cooling agents which are selected oxalic acid derivatives. The cooling system is stable, does not require special packaging and still provide a cooling sensation even after storage of the products.

### FIELD OF THE INVENTION

The present invention relates to a feminine care absorbent article comprising a cooling system.

### BACKGROUND OF THE INVENTION

Disposable absorbent articles, such as diapers, training pants, and catamenial devices having a cooling system are known. A cooling system typically consists of one or more cooling agents and can be introduced into the absorbent article alone or as components in a lotion composition. Cooling agents are able to stimulate thermo-receptors of the skin to convey freshness sensation to the wearer of the article, without the need of modifying body surface temperature.

Lotions of various types can be used to deliver the cooling system which are known to provide various additional skin benefits, such as prevention or treatment of diaper rash. These lotions can be applied to the topsheet of absorbent articles, for example, and can be transferred to the skin of the wearer during use. Alternatively a composition and or a lotion containing the cooling agents can be applied to inner portions of the absorbent articles such as the absorbent core, the secondary topsheet/acquisition layer or any other component of the absorbent article as long as the compositions can still be transferred to the skin of the wearer during use for example in consequence of movements, pressure, folding or insults from body fluids.

Certain wearers of disposable absorbent articles, such as catamenial devices, prefer products that can provide sensorial benefits, in addition to fluid absorbency benefits. A sensorial benefit desired by some wearers is a cooling sensation that can convey a feeling of freshness to the wearer. There has thus been a desire to create an absorbent article, such as a catamenial article which is able to deliver such a freshness feeling.

Previous attempts have been made to incorporate conventional cooling agents, such as menthol, menthyl lactate or N-ethyl p-menthanecarboxamide (also known as “WS-3”), in absorbent articles, such as catamenial devices. See, e.g., US 2004/00826654 A1 and US 2004/0081680 A1. Due to their volatile property, these materials are generally placed in products that can directly reach the sensory receptors in the skin or mucosal tissue. This is achieved either through direct contact with the target tissue (i.e., oral rinse, nasal spray, massage oil). In the context of an absorbent article these materials are generally loaded into the core of the product. Nonetheless, these conventional cooling agents remain highly volatile such that it can be difficult for such materials in an absorbent article to remain stable upon shipment and storage, especially when exposed to high temperatures during shipment and storage. Absorbent articles containing cooling agents exist in the market nowadays and are normally distributed in sealed packages which have strong barrier properties against the evaporation of the cooling agents, typically including an aluminum foil. These type of packages, while helping to preserve the cooling agent, add costs and complexity to the production and the distribution of the absorbent articles.

Other cooling agents have recently been identified that have lower volatility. However, this presents a problem of delivery to the epidermal cooling receptors. Furthermore, some of these cooling agents can be unacceptable to the wearer due to the odor of the cooling agent.

A desire thus remains to develop an absorbent article product that can provide a sensorial benefit to the wearer, such as a cooling sensation, which have acceptable odor and can be delivered to the skin of the wearer and which can be packaged using conventional packing materials and techniques without suffering from stability problems upon shipment and storage.

### SUMMARY OF THE INVENTION

The present invention relates to an absorbent article comprising a cooling system, said cooling system comprising a cooling agent which is an oxalic acid derivative having the formula

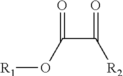

wherein R1 is a menthyl radical selected from menthyl, neomenthyl, isomenthyil, and mixtures thereof and R2 is independently selected from —O(CH3), —NH2, —NH(CH3), —N(CH3)2, —NH—(CH2)n-CH3, —NH—CN2-CH2-O—CH3 and mixture thereof wherein n is comprised between 1 and 15.

The absorbent articles of the present invention comprising such cooling agents provide a beneficial cooling sensation to the skin of the wearer of the absorbent article.

### DETAILED DESCRIPTION OF THE INVENTION

As used herein, the term “absorbent article” refers to devices that absorb and contain body exudates, such as menses, vaginal discharges or urine. The term “disposable” is used herein to describe absorbent articles which are not intended to be laundered or otherwise restored or reused as an absorbent article after a single use. Examples of absorbent articles include feminine hygiene garments such as sanitary napkins, pantyliners, interlabial devices, hemorrhoid pads, adult incontinence devices such as pads or briefs and baby diapers.

The absorbent articles herein are typically utilized by consumers who can detect and react to sensate materials, such as the cooling agents described herein, predominantly adults.

Disposable absorbent articles and components thereof, including the topsheet, backsheet, absorbent core, and any individual layers of these components, have a body surface and a garment surface. As used herein, “body surface” means that surface of the article or component which is intended to be worn toward or adjacent to the body of the wearer, while the “garment surface” is on the opposite side and is intended to be worn toward or placed adjacent to the wearer's undergarments when the disposable absorbent article is worn.

Typical absorbent articles for use herein are catamenial devices that can be a sanitary napkin or pantyliner, having a body-contacting surface comprising a topsheet, a liquid impervious backsheet joined to the topsheet, an absorbent core positioned in between. The sanitary napkin may also be provided with additional features commonly found in napkins, including “wings” or “flaps” as is known in the art, and, and/or a fluid acquisition layer (also known as secondary topsheet) positioned between topsheet and absorbent core to promote fluid transport and distribution to the absorbent core. Likewise, the topsheet of the sanitary napkin can have various optional characteristics, as is known in the art. For example, the topsheet can have channels embossed therein to direct fluid flow, can have apertures therethrough to aid in fluid acquisition. A lotion composition can be disposed for example onto the topsheet on its garment facing surface or on its wearer facing surface as well as on the wearer or garment facing surfaces of the secondary topsheet.

The topsheet is preferably compliant, soft feeling, and non-irritating to the wearers skin and hair. Further, the topsheet is liquid pervious, permitting liquids (e.g., menses and/or urine) to readily penetrate through its thickness. A suitable topsheet may be manufactured from a wide range of materials such as woven and nonwoven materials (e.g., a nonwoven web of fibers); polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the topsheet comprises a nonwoven web, the web may be manufactured by a wide number of known techniques. For example, the web may be spunbonded, carded, wet-laid, melt-blown, hydroentangled, combinations of the above, or the like.

The backsheet is typically impervious to liquids (e.g., menses and/or urine) and is preferably manufactured from a now woven or a thin plastic film, although other flexible liquid impervious materials may also be used. As used herein, the term “flexible” refers to materials which are compliant and will readily conform to the general shape and contours of the human body. The backsheet prevents the exudates absorbed and contained in the absorbent core from wetting articles which contact the absorbent article such as bedsheets, pants, pajamas and undergarments. The backsheet may thus comprise a woven or nonwoven material, polymeric films such as thermoplastic films of polyethylene or polypropylene, or composite materials such as a film-coated nonwoven material.

The absorbent core can be joined with the topsheet, the backsheet, or both in any manner as is known by attachment means such as those well known in the art. However, embodiments of the present invention are envisioned wherein portions of the entire absorbent core are unattached to either the topsheet, the backsheet, or both.

In one embodiment, the topsheet of catamenial device is made of a hydrophobic material. Therefore, if the topsheet is a nonwoven, the constituent fibers are preferably hydrophobic. Fibers are considered to be hydrophobic if film sheets formed from the polymers of the fibers would exhibit contact angles with water greater than 60 degrees, more preferably 75 degrees, and even more preferably greater than about 90 degrees. Contact angles as a measure of hydrophobicity are well known in the art, and methods for measuring contact angles are equally well known. As is well known, contact angles greater than about 90 degrees are considered hydrophobic, and contact angles less than 90 degrees are often considered hydrophilic. As used herein, however, contact angles of 60 degrees or greater are considered hydrophobic.

In another embodiment, the topsheet of catamenial device is made of a hydrophilic material.

The absorbent core can be formed from any of the materials well known to those of ordinary skill in the art. Examples of such materials include multiple plies of creped cellulose wadding, fluffed cellulose fibers, wood pulp fibers also known as airfelt, textile fibers, a blend of fibers, a mass or batt of fibers, airlaid webs of fibers, a web of polymeric fibers, and a blend of polymeric fibers. Other suitable absorbent core materials include absorbent foams such as polyurethane foams or high internal phase emulsion (“HIPE”) foams. Suitable HIPE foams are disclosed in U.S. Pat. No. 5,550,167, U.S. Pat. No. 5,387,207, U.S. Pat. No. 5,352,711, and U.S. Pat. No. 5,331,015.

For some absorbent articles, the absorbent core can be relatively thin, less than about 5 mm in thickness, or less than about 3 mm, or less than about 1 mm in thickness. Thickness can be determined by measuring the thickness at the midpoint along the longitudinal centerline of the pad by any means known in the art while under a uniform pressure of 1.72 kPa.

The absorbent core can comprise superabsorbent materials such as absorbent gelling materials (AGM), including AGM fibers, as is known in the art. The absorbent core can therefore constitute a layer comprising superabsorbent material.

The absorbent articles of the present invention comprise a cooling system which comprises an oxalic acid derivative having the formula

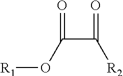

wherein R₁ is a menthyl radical selected from menthyl, neomenthyl, isomenthyil, and R₂ is independently selected from —O(CH₃), —NH₂, —NH(CH₃), —N(CH₃)₂, —NH—(CH₂)ₙ—CH₃, —NH—CH₂—CH₂—O—CH₃ (wherein n is comprised between 1 and 15).
All possible stereoisomers are included in the scope of the compounds represented by the above formula, although in some preferred embodiments the R1 radicals, when a D and an L form are possible, are predominantly or entirely in L form
Some exemplary embodiments of cooling agents which are suitable for use in the present inventions are the following: R₁ is “L-menthyl” and R₂ is “methoxy” i.e. “—O—CH3”.

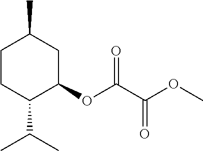 [SMILES: COC(=O)C(=O)O[C@@H]1C[C@H](C)CC[C@H]1C(C)C]

R₁ is “L-menthyl” and R₂ is “methylamino”.

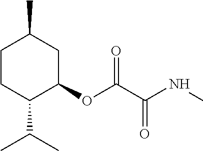 [SMILES: CNC(=O)C(=O)O[C@@H]1C[C@H](C)CC[C@H]1C(C)C]

R₁ is “L-menthyl” and R₂ is “ethylamino”.

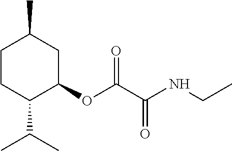 [SMILES: CCNC(=O)C(=O)O[C@@H]1C[C@H](C)CC[C@H]1C(C)C]

R₁ is “neomenthyl” and R₂ is “methylamino”.

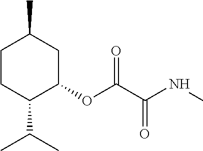 [SMILES: CNC(=O)C(=O)O[C@H]1C[C@H](C)CC[C@H]1C(C)C]

R₁ is “L-menthyl” and R₂ is “amino”.

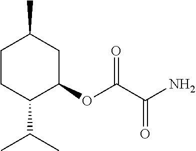 [SMILES: CC(C)[C@@H]1CC[C@@H](C)C[C@H]1OC(=O)C(N)=O]

R₁ is “D-menthyl” and R₂ is “dimethyl amino”.

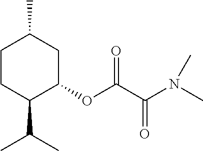 [SMILES: CC(C)[C@H]1CC[C@H](C)C[C@@H]1OC(=O)C(=O)N(C)C]

These cooling agents are known and their synthesis is described for example in US2009/0054520A1.

As mentioned above, most cooling agents are difficult to use into absorbent articles because absorbent articles are commonly packaged using low thickness plastic wraps and are shipped and stored for months in the most different temperature conditions.

The most effective and common cooling agents (such as menthol) are too volatile to be compatible with these conditions, and lose their activity in a few weeks or even in a few days if exposed to high storage temperatures. Menthol has been commercially employed by using extremely expensive and complex packages employing thick plastic film and aluminum foils which provided better sealing and protection from evaporation. This however adds costs and complexity to the manufacturing line and is therefore a solution which can be further improved.

Alternative less volatile cooling agents have been identified, such as menthyl lactate, which offer an improved tradeoff between volatility and efficacy however their stability is still not optimal if the absorbent articles are stored for long periods and in warehouses which can be at high temperatures such at 35 or 40° C. during summer months as desired by the industry. Therefore there is still a demand for effective cooling agents which can be easily introduced into absorbent articles as pure materials or as components of lotions, which are stable enough to withstand long term storage in any condition and which are still effective at the time of usage even on aged products.

The cooling system of the present invention is an effective solution to this problem thanks to the presence of the selected cooling agents mentioned above. These cooling agents have been selected to have a surprisingly high resistance to evaporation when incorporated into an absorbent article and at the same time a surprisingly high capacity to provide a cooling sensation upon product usage.

In the cooling system of the present invention the particular cooling agents, such as the oxalic acid derivatives recited above can be utilized alone, in combination with each other, and/or in combination with more traditional cooling materials known in the art, for example, menthol, menthone, isopulegol, N-ethyl p-menthanecarboxamide (WS-3), N,2,3-trimethyl-2-isopropylbutanamide (WS-23), menthyl lactate (Frescolat® ML), menthone glycerine acetal (Frescolat® MGA), mono-menthyl succinate (Physcool®), mono-menthyl glutarate, O-menthyl glycerine (CoolAct® 10), menthyl-N,N-dimethylsuccinamate or 2-sec-butylcyclohexanone) (Freskomenthe®.

The cooling system of the present invention can be used alone (e.g. applying the mixture directly onto a component of the absorbent article) or as a component of a lotion comprising other ingredients such as a carrier and or skin benefit agents. When comprised in a lotion, the cooling system will typically be present at a concentration ranging from about 0.0001% to about 90.0%, from about 0.0001% to about 30.0%, from about 0.001% to about 10.0%, or from about 0.01% to about 1.0%, by weight of the lotion composition. The cooling system can also be prepared in a premix in an oil diluents such premix can be used alone or introduced into a lotion.

If the cooling system of the present invention is formulated in a lotion, such lotion typically comprises a carrier or a mixture of carriers. The carrier helps to deliver the cooling agents of the present invention to the skin of the wearer of the absorbent article. The carrier can be included in the compositions as an individual carrier or a combination of carrier ingredients. The carrier can be a liquid, solid, or semisolid carrier material, or a combination of these materials, and preferably forms a homogenous mixture or solution at selected processing temperatures for the resultant carrier system and at processing temperatures for combining the carrier with the cooling agents in formulating the lotion compositions herein. Processing temperatures for the carrier system typically range from about 60° C. to about 90° C., more typically from about 70° C. to about 85° C., even more typically from about 65° C. to about 80° C.

The lotion compositions can comprise the carrier at a total carrier concentration ranging from about 60% to about 99.9%, preferably from about 70% to about 98%, more preferably from about 80% to about 97%, by weight of the lotion composition. Suitable carrier compounds include petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin and its derivatives, glyceride and its derivatives including acetoglycerides and ethoxylated glycerides of C12-C28 fatty acids, and mixtures thereof. Alternatively or in combination with, the carrier may also be composed of polysiloxane compounds non-limiting examples include dimethicones (1-100,000,000 centistoke), cyclomethicones, alkylated silicones (hair conditioning agents), silicone gums, silicone gels, silicone waxes, copolymers of silicone (vinyl dimethicone polymers, phenyl vinyl dimethicone polymers, alkylated silicone polymers, polyethylene oxide/silicone copolymers, polyethylene oxide/alkyl silicone copolymers), and mixtures thereof.

Nonlimiting examples of suitable petroleum-based hydrocarbons having from about 4 to about 32 carbon atoms include mineral oil, petrolatum, isoparaffins, various other branched chained hydrocarbons, and combinations thereof. Mineral oil is also known as “liquid petrolatum”, and usually refers to less viscous mixtures of hydrocarbons having from about 16 to about 20 carbon atoms. Petrolatum is also known as “mineral wax”, “petroleum jelly”, and “mineral jelly”, and usually refers to more viscous mixtures of hydrocarbons having from about 16 to about 32 carbon atoms. An example of commercially available petrolatum include petrolatum sold as Protopet® 1S which is available from the Witco Corporation located in Greenwich, Conn.

Other carriers suitable herein can include oils or fats such as natural oils or fats, or natural oil or fat derivatives, in particular of plant or animal origin. Non-limiting examples include avocado oil, apricot oil, apricot kernel oil, babassu oil, borage oil, borage seed oil, calendula oil, camellia oil, canola oil, carrot oil, cashew nut oil, castor oil, chamomile oil, cherry pit oil, chia oil, coconut oil, cod liver oil, corn oil, corn germ oil, cottonseed oil, eucalyptus oil, evening primrose oil, grape seed oil, hazelnut oil, jojoba oil, juniper oil, kernel oil, linseed oil, macadamia oil, meadowfoam seed oil, menhaden oil, mink oil, moringa oil, mortierella oil, olive oil, palm oil, palm kernel oil, peanut oil, peach kernel oil, rapeseed oil, rose hip oil, safflower oil, sandlewood oil, sesame oil, soybean oil, sunflower oil, sunflower seed oil, sweet almond oil, tall oil, tea tree oil, turnip seed oil, walnut oil, wheat germ oil, zadoary oil, or the hardened derivatives thereof. Hardened oils or fats from vegetal origin can include, e.g. hardened castor oil, peanut oil, soya oil, turnip seed oil, cottonseed oil, sunflower oil, palm oil, kernel oil, linseed oil, corn oil, olive oil, sesame oil, cocoa butter, shea butter and coconut oil.

Other non-limiting examples of fats and oils include: butter, C12-C18 acid triglyceride, camellia oil, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, human placental lipids, hybrid safflower oil, hybrid sunflower seed oil, hydrogenated castor oil, hydrogenated castor oil laurate, hydrogenated coconut oil, hydrogenated cottonseed oil, hydrogenated C12-C18 triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lanolin and lanolin derivatives, lanolin alcohol, lard, lauric/palmitic/oleic triglyceride, lesquerella oil, maleated soybean oil, meadowfoam oil, neatsfoot oil, oleic/linoleic triglyceride, oleic/palmitic/lauric/myristic/linoleic triglyceride, oleostearine, olive husk oil, omental lipids, orange roughy oil, pengawar djambi oil, pentadesma butter, phospholipids, pistachio nut oil, placental lipids, rapeseed oil, rice bran oil, shark liver oil, shea butter, sphingolipids, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, wheat bran lipids, and the like, as well as mixtures thereof.

Other suitable carriers include mono- or di-glycerides, such as those derived from saturated or unsaturated, linear or branch chained, substituted or unsubstituted fatty acids or fatty acid mixtures. Examples of mono- or diglycerides include mono- or di-C₁₂₋₂₄ fatty acid glycerides, specifically mono- or di-C₁₆₋₂₀ fatty acid glycerides, for example glyceryl monostearate, glyceryl distearate.

Carriers can also include esters of linear C₆-C₂₂-fatty acids with branched alcohols. The carrier can also include sterols, phytosterols, and sterol derivatives. Sterols and sterol derivatives that can be used in the lotion compositions include, but are not limited to: β-sterols having a tail on the 17 position and having no polar groups for example, cholesterol, sitosterol, stigmasterol, and ergosterol, as well as, C10-C30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A commercially available example of phytosterol is GENEROL 122 N PRL refined soy sterol from Cognis Corporation of Cincinnati, Ohio.

Nonlimiting examples of suitable fatty alcohols having from about 12 to about 24 carbon atoms include saturated, unsubstituted, monohydric alcohols or combinations thereof, which have a melting point less than about 110° C., preferably from about 45° C. to about 110° C. Specific examples of fatty alcohol carriers for use in the lotion compositions include, but are not limited to, cetyl alcohol, stearyl alcohol, cetearyl alcohol, behenyl alcohol, arachidyl alcohol, lignocaryl alcohol, and combinations thereof. Examples of commercially available cetearyl alcohol is Stenol 1822 and behenyl alcohol is Lanette 22, both of which are available from the Cognis Corporation located in Cincinnati, Ohio.

Nonlimiting examples of suitable fatty acid esters include those fatty acid esters derived from a mixture of C₁₂-C₂₈ fatty acids and short chain (C₁-C₈, preferably C₁-C₃) monohydric alcohols preferably from a mixture of C₁₆-C₂₄ saturated fatty acids and short chain (C₁-C₈, preferably C₁-C₃) monohydric alcohols. Suitable fatty acid esters can also be derived from esters of longer chain fatty alcohols (C₁₂-C₂₈, preferably C₁₂-C₁₆) and shorter chain fatty acids such as lactic acid, specific examples of which include lauryl lactate and cetyl lactate. Representative examples of suitable fatty acid esters include methyl palmitate, methyl stearate, isopropyl laurate, isopropyl myristate, isopropyl palmitate, ethylhexyl palmitate, stearyl stearate, palmityl stearate, stearyl behenate, cetyl stearate, cetyl behenate, cetyl palmitate, cetearyl behenate, behenyl behenate, stearyl heptanoate, stearyl octanoate, myristyl myristate, myristyl isostearate, myristyl oleate, cetyl isostearate, cetyl oleate, stearyl isostearate, stearyl oleate, isostearyl myristat, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl isostearate, behenyl oleate, erucyl isostearate, and mixtures thereof.

Nonlimiting examples of suitable alkyl ethoxylates include C₁₂-C₂₂ fatty alcohol ethoxylates having an average degree of ethoxylation of from about 2 to about 30. Nonlimiting examples of suitable lower alcohols having from about 1 to about 6 carbon atoms include ethanol, isopropanol, butanediol, 1,2,4-butanetriol, 1,2 hexanediol, ether propanol, and mixtures thereof. Nonlimiting examples of suitable low molecular weight glycols and polyols include ethylene glycol, polyethylene glycol (e.g., Molecular Weight 200-600 g/mole), butylene glycol, propylene glycol, polypropylene glycol and mixtures thereof. A more detailed description of carrier ingredients including suitable hydrocarbons, polysiloxane compounds, and fatty alcohol ethoxylates can be found in U.S. Pat. No. 5,643,588, issued Jul. 1, 1997 to Roe et al. entitled “Diaper Having A Lotioned Topsheet”.

Suitable carriers further encompass waxes. As used herein, the term ‘wax’ refers to oil soluble materials that have a waxy constituency and have a melting point or range of above ambient temperature, in particular above 25° C. Waxes are materials that have a solid to semisolid (creamy) consistency, crystalline or not, being of relative low viscosity a little above their liquefying point. Suitable waxes which can be incorporated into the lotion composition include animal, vegetable, mineral or silicone based waxes which may be natural or synthetic, and including mixtures thereof. Waxes can include but are not limited to: natural waxes from vegetal origin, such as bayberry wax, beeswax, candelilla wax, carnauba, ceresin, purcelline, shea butter, cocoa butter, Japan wax, jojoba wax, lanolin wax, esparto gras wax, cork wax, guaruma wax, rice shoot wax, ouricury wax, mink wax, montan wax, rice bran wax, spent grain wax, spermaceti wax, steryl dimethicone, sunflower wax, ceresine wax, sugar cane wax, carnauba wax, candelilla wax, fruit-derived waxes, such as orange wax, lemon wax, grapefruit wax and bayberry wax, and the like; and waxes from animal origin such as beeswax, woolwax, bear fat, shellac wax, and the like. Natural waxes further comprise mineral waxes such as ceresin and ozokerite waxes. Synthetic waxes comprise petroleum-based waxes, such as certain carrier materials described hereinbefore, such as paraffin, vaseline, petrolatum, micro wax, and microcrystalline wax. Further suitable synthetic waxes are polyalkylene and polyethyleneglycol waxes, e.g. polyethylene wax; waxes based on chlorinated naphtalenes such as ‘Halowax’, synthetic hydrocarbon waxes, and the like, PEG-6 beeswax, PEG-8 beeswax, C30 alkyl dimethicone, synthetic beeswax, synthetic candelilla wax, synthetic carnuba wax, synthetic japan wax, synthetic jojoba wax, motan acid wax, motan wax, ouricury wax, rezowax, including mixtures thereof. Further suitable waxes are chemically modified waxes, in particular hardened or hydrogenated waxes such as, for example, hydrogenated cottonseed oil, hydrogenated jojoba oil, hydrogenated jojoba wax, hydrogenated microcrystalline wax, hydrogenated rice bran wax, Montan-ester waxes, Sasol waxes, jojoba esters, and the like.

Other wax components can be certain fats (including mono-, di- and triglycerides and fatty acid alkylesters), fatty alcohols, fatty acids, including substituted fatty acids (in particular hydroxy substituted fatty acids, for example, 12-hydroxystearic acid), dialkyl(ene)ethers, dialkyl(ene) carbonates, dicarboxylic acids (in particular the C₁₆-C₄₀-dialkylesters of dicarboxylic acids, e.g. the C₁₆-C₄₀-alkyl stearates, C₁₈-C₃₈-alkylhydroxystearyl stearates or C₂₀-C₄₀-alkyl erucates) and hydroxy fatty alcohols. Still further wax components are selected from the group of aromatic carbonic acids, tricarboxylic acids, or from the group of lactides of long-chained hydroxycarbonic acids. Myristyl lactate is a suitable carrier. Further wax components that can be used are C₃₀-C₅₀ alkyl bees wax; tri-C₁₆-C₄₀-alkyl citrates, e.g. tristearyl citrate, triisostearyl citrate, trilauryl citrate; ethyleneglycol di fatty acid esters, in particular the ethylene glycol di-C₁₂-C₃₀-fatty acid esters, e.g. ethylene glycol dipalmitate, ethyleneglycol distearate, and ethyleneglycol di(12-hydroxystearate).

Other suitable carriers include materials that act as solidifying agents, including some of the materials described hereinbefore. Suitable solidifying agent(s) in the lotion compositions of the present invention can function to help solidify the composition so that the composition is a solid at room temperature and has a melting point of at least 32° C. The solidifying agent may also provide a tackiness to the composition that improves the transfer by adhesion to the skin of the wearer. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer and combinations thereof. The solidifying agents can be selected from alkyl siloxanes, polymers, hydrogenated vegetable oils having a melting point of 35° C. or greater, fatty acid esters with a melting point of 35° C. or greater, alkyl hydroxystearates, branched esters, alkoxylated alcohols and alkoxylated carboxylic acid. Additionally, the solidifying agents can be selected from animal, vegetable and mineral waxes and alkyl silicones. Examples of suitable solidifying agents include, but are not limited to, the following: alkyl silicones, alkyl trimethylsilanes, beeswax, behenyl behenate, behenyl benzoate, C24-C28 alkyl dimethicone, C30 alkyl dimethicone, cetyl methicone, stearyl methicone, cetyl dimethicone, stearyl dimethicone, cerotyl dimethicone, candelilla wax, carnuba, synthetic carnuba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C20-C40 alkyl behenate, C2-05 lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di C2-C5 alkyl fumerate, dibehenyl fumerate, myristyl lactate, myristyl lignocerate, myristyl myristate, myristyl stearate, lauryl stearate, octyidodecyl stearate; octyidodecyl stearoyl stearate, oleyl arachidate, oleyl stearate, tridecyl behenate, tridecyl stearate, tridecyl stearoyl stearate, pentaerythrityl tetrabehenate, penteerythritylhydrogenated rosinate, pentaerythrityl distearate, pentaerythrityltetraabeite, penteerythrityl tetracocoate, penteerythrityl tetraperlargonate, pentserythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C30-C60 fatty alcohols, C20+ fatty amides, polypropylene, polystyrene, polybutane, polybutylene terephthalate, polydipentane, polypropylene, zinc stearate, dodecyl laurate, stearyl palmitate, octadecyl hexedecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C20-C40 alkyl hydroxystearyl stearate, and mixtures of such compounds.

In a preferred embodiment of the invention the carrier comprises a polyethylene glycol or a mixture of different polyethylene glycols, wherein the polyethylene glycol(s) has/have a weight average molecular weight Mw of 2000 Da or more.

The lotion composition herein can further optionally comprise a skin care composition comprising a combination of skin treatment agents such as hexamidine, zinc oxide, and niacinamide.

Hexamidine skin treatment agent can be comprised at concentrations ranging from about 0.001% to about 0.1%, preferably from about 0.005% to about 0.1%, more preferably from about 0.01% to about 0.1% by weight of the composition.

Niacinamide skin treatment agent can be comprised as an individual niacinamide or as a combination of niacinamides at a total niacinamide concentration ranging from about 0.01% to about 10%, preferably from about 0.05% to about 5%, more preferably from about 0.2% to about 2% by weight of the skin care composition. The niacinamide skin treatment agent provides for skin conditioning benefits as well as providing for increased efficacy of the skin treatment agents in controlling skin disorders.

Zinc oxide skin treatment agent can be comprised at concentrations ranging from about 0.001% to about 30%. The zinc oxide skin treatment agent can be included in the compositions as an individual zinc oxide compound or a combination of zinc oxides.

It has been noticed that in some conditions the presence in the lotion of Zn containing compounds, especially Zinc oxide, can reduce the stability of some of the cooling agents of the present invention. While the mechanism has not been fully understood, it is in general preferred that the lotion compositions of the present invention have a low content of Zinc. In some embodiments lotions according to the present invention comprise less than 5% or less than 2% or less than 1% or less than 0.5% or less than 0.1% of Zinc Oxide by weight of the total weight of the lotion. In another embodiment the lotion according to the present invention is free of Zinc Oxide.

The absorbent article of the present invention can optionally further comprise indicia to emphasize the cooling benefits or cooling sensation provided by the absorbent article. Such indicia can include graphics, patterns, and the like that comprise colors such as green, blue, or combinations thereof. The colors green, blue, or combinations thereof tend to visually communicate the cooling benefits or cooling sensation provided by the absorbent article to the consumer. Such indicia can be incorporated into the packaging for the absorbent articles or can be incorporated into the absorbent articles themselves, such as indicia that is printed or impregnated into the components of the absorbent article (e.g. the topsheet or backsheet of the absorbent article).

The absorbent article of the present invention can optionally further comprise essential oil materials that help to connote the cooling benefits or cooling sensation provided by the absorbent article. Such essential oil materials can be incorporated into the absorbent article separate from the lotion composition or can be made part of the lotion composition. Non-limiting examples of suitable essential oil materials include Acorns gramineus, Anthemis nobilis, Artemisia dracunculus, Basil, Bergamot, Calamintha sylvatica, Caraway, Cedarwood, Chamomile, Cineol, Cinnamon, Cinnamon bark, Citrus aurantium, Clove, Cypress, Dill, Eucalyptus, Eugenol, Frankincense, Galangol, Geranium, Ginger, Hibiscus, Hop, Jasmine, Juniper, Laurus nobilis, Lavender, Lemon balm, Lemongrass, Lemon, Limonene, Linalool, Linalyl acetate, Lippia Alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, Hibiscus, or mixtures thereof. Preferred essential oils associated with arousal include Cypress, Hibiscus, Juniper, Cineol, Citrus, Sweet Orange, and Rosemary. Preferred oils associated with a harmonizing effect include Lavender, Neroli, and Ylang ylang.

The particular essential oils herein, such as described above, can be blended in a carrier at a concentration ranging from about 0.0001% to about 10.0%, from about 0.0001% to about 3.0%, from about 0.0001% to about 0.1%, from about 0.001% to about 1%, or from about 0.01% to about 1.0%, by weight of the lotion composition. The essential oil can also be prepared in a premix in an oil diluent. Nonetheless, the final concentration of the essential oil will typically fall in the ranges described above.

The cooling agents of the present invention, other ingredients of the lotion composition, or the lotion composition itself can be encapsulated in a variety of suitable encapsulation materials. Non-limiting examples of suitable encapsulation materials are described in detail as “Perfume Carrier Materials” in U.S. Pat. No. 7,186,680 at column 25, line 23 to column 28, line 52.

When applied to the outer surface of sanitary napkin topsheets, the lotion compositions of the present invention can be transferable to the wearer's skin by normal contact, wearer motion, and/or body heat, thereby providing a cooling sensation on the skin of the wearer.

In some embodiments the topsheets of the absorbent articles of the present invention contain an effective amount of the lotion composition. As used herein, the term “effective amount of a lotion composition” refers to an amount of a particular lotion composition which, when applied to a sanitary napkin topsheet, will be effective in providing a cooling benefit or cooling sensation to the skin of the wearer. The effective amount of a lotion composition will depend, to a large extent, on the particular lotion composition used.

In preparing lotioned absorbent articles according to the present invention, the lotion composition can be applied, as mentioned above, to any other component of the absorbent article. In some embodiments the lotion is applied to the outer surface (i.e., body facing surface) of the topsheet, or to the inner surface of the topsheet or to the body facing or garment facing surface of the secondary topsheet or to the body facing surface of the absorbent core.

Any of a variety of application methods that evenly distribute the lotion composition can be used. Suitable methods include spraying, printing (e.g., flexographic printing), coating (e.g., gravure coating), extrusion, or combinations of these application techniques, e.g. spraying the lotion composition on a rotating surface, such as a calender roll, that then transfers the composition to the outer surface of the topsheet.

The manner of applying the lotion composition to a component of the article, can be such that the component does not become saturated with the lotion composition. If the component is e.g. the topsheet and it becomes saturated with the lotion composition, there is a greater potential for the lotion to block the topsheet openings, reducing the ability of the topsheet to transmit fluid to the underlying absorbent core. Also, saturation of the topsheet is not required to obtain the therapeutic and/or protective lotion benefits. Particularly suitable application methods will apply the lotion composition primarily to the outer surface of the topsheet.

The minimum level of lotion to be applied to the topsheet is an amount effective for providing a cooling benefit or cooling sensation to the skin of the wearer. The lotion composition is preferably applied to the topsheet of the present invention in an amount ranging from about 0.1 mg/in² to about 75 mg/in² (mg of lotion per square inch of coated topsheet), preferably from about 0.5 mg/in² to about 60 mg/in², and more preferably from about 1 mg/in² to about 60 mg/in². Such relatively low levels of lotion composition are adequate to impart the desired cooling benefits, yet do not saturate the topsheet's absorbency and/or wettability properties.

The lotion composition may be applied to the entire surface of the topsheet or portions thereof. The lotion composition can be applied in a stripe aligned with and centered on the longitudinal centerline of the disposable absorbent article. The lotion composition can be applied in a plurality of stripes having uniform or non-uniform widths. Alternatively the lotion can be aligned with and centered in opposition to the longitudinal centerline.

The lotion composition can also be applied nonuniformly to the outer surface of the sanitary napkin topsheet. By “nonuniform” is meant that the amount, pattern of distribution, etc. of the lotion composition can vary over the topsheet surface. For example, some portions of the treated surface of the topsheet can have greater or lesser amounts of lotion composition, including portions of the surface that do not have any lotion composition on it. For example, the lotion composition can be applied on one region of the topsheet in the shape of a rectangle and/or a circle, and/or as mutliplicity of dots.

The lotion composition can be applied to the topsheet or other component at any point during assembly. For example, the lotion composition can be applied to the topsheet of the finished disposable absorbent product before it has been packaged. The lotion composition can also be applied to the topsheet before it is combined with the other raw materials to form a finished disposable absorbent product.

The lotion composition is typically applied from a melt thereof to the absorbent article. Since the lotion composition will typically melt at significantly above ambient temperatures, it is usually applied as a heated coating. Typically, the lotion composition is heated to a temperature in the range from about 35° C. to about 100° C., preferably from 40° C. to about 90° C., prior to being applied. Once the melted lotion composition has been applied, it is allowed to cool and solidify to form solidified coating or film on the surface of the topsheet or other component. Preferably, the application process is designed to aid in the cooling/set up of the lotion.

Lotion compositions can be applied by printing methods, or continuous spray or extrusion as is known in the art, or as is described in U.S. Pat. No. 5,968,025.

It can be preferred that the lotion be applied in a plurality of stripes parallel to the longitudinal axis of the absorbent article. This allows for both transfer of the lotion to a broader area of the vulva and improved fluid handling of the absorbent article.

In another embodiment, instead of (or in addition to) being applied to the topsheet of an absorbent article, the lotion composition can be provided as a stand-alone product in the form of a cream product that can be applied to the absorbent article by hand. In another embodiment, the lotion composition can be provided as a stand-alone product in the form of a spray product that can be sprayed onto the absorbent article by the wearer of the absorbent article.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a cooling system, said cooling system comprising a cooling agent which is an oxalic acid derivative having the formula 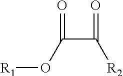
wherein R₁ is a menthyl radical selected from menthyl, neomenthyl, isomenthyil, and mixtures thereof
and R₂ is independently selected from —O(CH₃), —NH₂, —NH(CH₃), —N(CH₃)₂, —NH—(CH₂)ₙ—CH₃, —NH—CN2-CH2-O—CH3 and mixture thereof wherein n is comprised between 1 and 15.

2. The absorbent article of claim 1 wherein said cooling system comprises more than one of said oxalic acid derivatives.

3. The absorbent article of claim 1 wherein the oxalic acid derivative is selected from the following compounds:
i) R₁ is “L-menthyl” and R₂ is “methoxy” i.e. “—O—CH3”. 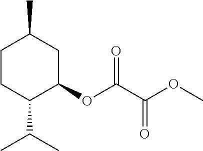 [SMILES: COC(=O)C(=O)O[C@@H]1C[C@H](C)CC[C@H]1C(C)C]
ii) R₁ is “L-menthyl” and R₂ is “methylamino”. 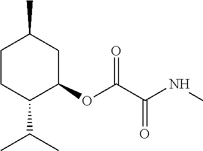 [SMILES: CNC(=O)C(=O)O[C@@H]1C[C@H](C)CC[C@H]1C(C)C]
iii) R₁ is “L-menthyl” and R₂ is “ethylamino”. 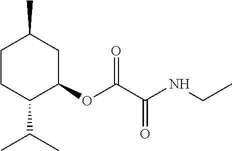 [SMILES: CCNC(=O)C(=O)O[C@@H]1C[C@H](C)CC[C@H]1C(C)C]
iv) R₁ is “neomenthyl” and R₂ is “methylamino”. 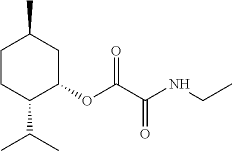 [SMILES: CCNC(=O)C(=O)O[C@H]1C[C@H](C)CC[C@H]1C(C)C]
v) R₁ is “L-menthyl” and R₂ is “amino”. 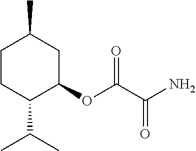 [SMILES: CC(C)[C@@H]1CC[C@@H](C)C[C@H]1OC(=O)C(N)=O]
vi) R₁ is “D-menthyl” and R₂ is “dimethyl amino”. 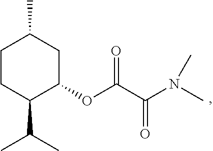 [SMILES: CC(C)[C@H]1CC[C@H](C)C[C@@H]1OC(=O)C(=O)N(C)C]
and mixture thereof.

4. The absorbent article of claim 1 wherein said cooling system in comprised with a lotion said lotion comprising a carrier.

5. The absorbent article of claim 4, wherein said carrier is selected from the group consisting of petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin, lanolin derivatives, glycerides, glyceride derivatives including acetoglycerides and ethoxylated glycerides of C₁₂-C₂₈ fatty acids, and mixtures thereof.

6. The absorbent article of claim 1, wherein said carrier comprises petrolatum.

7. The absorbent article of claim 4 wherein said lotion comprises less than about 2% by weight of the lotion of zinc oxide.

8. The absorbent article of claim 1, wherein in said oxalic acid derivatives said methyl radicals R₁ are predominantly in L configuration.

9. The absorbent article of claim 1, wherein said article further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

10. The absorbent article of claim 1, wherein said cooling system is substantially odorless.

11. A method of providing a cooling sensation to the user of an absorbent article comprising the step of putting in contact with the skin of said user an absorbent article according to any preceding claim.

12. The use of an oxalic acid derivative having the formula 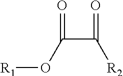
wherein R₁ is a menthyl radical selected from menthyl, neomenthyl, isomenthyil, and mixtures thereof
and R₂ is independently selected from —O(CH₃), —NH₂, —NH(CH₃), —N(CH₃)₂, —NH—(CH₂)ₙ—CH₃, —NH—CN2-CH2-O—CH3 and mixture thereof wherein n is comprised between about 1 and about 15,
within an absorbent article in order to provide a cooling sensation to the user of said absorbent article.
